SEQUENCE LISTING (1 sequence)

SEQ ID NO: 1 - protein, 325 aa, Artificial Sequence
  recombinant protein
AGGCGHAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLSGEDD

(19) United States
(12) Patent Application Publication
(10) Pub. No.: US 2022/0031872 A1
(43) Pub. Date: Feb. 3, 2022

(54) Lyophilized Compositions Comprising Rhannexin V-128, Process for Their Preparation and Their Use for Preparing Formulations Containing 99MTc-Rhannexin V-128

(71) Applicant: Advanced Accelerator Applications International S.A., Geneva (CH)

(72) Inventors: Lorenza Fugazza, Ivrea (IT); Maurizio Franco Mariani, Ivrea (IT); Francesca Orlandi, Ivrea (IT); Daniela Chicco, Albiano D'Ivrea (IT); Donato Barbato, Ivrea (IT)

(73) Assignee: Advanced Accelerator Applications International S.A., Geneva (CH)

(21) Appl. No.: 17/398,195

(22) Filed: Aug. 10, 2021

Related U.S. Application Data
(63) Continuation of application No. 16/339,530, filed on Apr. 4, 2019.

(30) Foreign Application Priority Data
Oct. 11, 2016 (IT) 102016000101875

(51) Int. Cl. A61K 51/08; A61K 9/00; A61K 9/19; A61K 47/02; A61K 47/12; A61K 47/26; A61K 47/40

(52) U.S. Cl. CPC A61K 51/087; A61K 9/0019; A61K 9/19; A61K 47/40; A61K 47/12; A61K 47/26; A61K 47/02

(57) ABSTRACT

It is described a composition comprising lyophilized rhAnnexin V-128 suitable for the preparation of 99mTc-rhAnnexin V-128 formulations suitable for intravenous administration.

### SEQUENCE LISTING SUBMISSION VIA EFS-WEB

A computer readable text file, entitled “SequenceListing.txt” created on or about Apr. 4, 2019 with a file size of about 4 kb contains the sequence listing for this application and is hereby incorporated by reference in its entirety.

### FIELD OF THE INVENTION

The present invention refers to labelled compounds in particular to Technetium labelled recombinant protein Annexin V-128.

### STATE OF THE ART

rhAnnexin-V-128 is a known recombinant protein having sequence (SEQ ID NO 1):

AGGCGHAQVLRGTVTDFPGFDERADAETLRKAMKGLGTDEESILT
LLTSRSNAQRQEISAAFKTLFGRDLLDDLKSELTGKFEKLIVALM
KPSRLYDAYELKHALKGAGTNEKVLTEIIASRTPEELRAIKQVYE
EEYGSSLEDDVVGDTSGYYQRMLVVLLQANRDPDAGIDEAQVEQD
AQALFQAGELKWGTDEEKFITIFGTRSVSHLRKVFDKYMTISGFQ
IEETIDRETSGNLEQLLLAVVKSIRSIPAYLAETLYYAMKGAGTD
DHTLIRVMVSRSEIDLFNIRKEFRKNFATSLYSMIKGDTSGDYKK
ALLLLSGEDD

This recombinant protein is described in Jin M. et al. “Essential Role of B-helix Calcium Binding Sites in Annexin V-Membrane Binding” The Journal of Biological Chemistry—Vol. 279—No. 39—pp. 40351-40357 (2004) and is a mutant form of Annexin V, a naturally occurring human serum protein, in which six amino acids (Ala-Gly-Gly-Cys-Gly-His) have been added to the N-terminus of rhAnnexin V (identical to wild type human Annexin V) to form an endogenous ^{99m}Tc-binding site.

In addition, cysteine in position 316 has been mutated to a serine (see the underlined S in the sequence above); with this mutation, the only cysteine remaining in the rhAnnexin V-128 sequence is the one in position 4, in the N-terminal ^{99m}Tc-binding site.

rhAnnexin V-128 is produced by known recombinant techniques in Escherichia coli (See for example Jin M. et al. “Essential Role of B-helix Calcium Binding Sites in Annexin V-Membrane Binding” The Journal of Biological Chemistry—Vol. 279—No. 39—pp. 40351-40357 (2004)) and it is stored in frozen form.

As said above the modification introduced in the N-terminus allows the binding of the protein with ^{99m}Tc to form the corresponding labelled protein that, thanks to its mechanism of action, has a broad spectrum of potential applications both as a diagnostic tool, as well as for monitoring treatment efficacy and is normally used by intravenous administration.

The most interesting indications are in the field of rheumatology, cardiovascular diseases, oncology, transplant rejection, autoimmune diseases, neurology, but there is room also for other pathologies having as a hallmark the process of apoptosis. With regard to cardiovascular diseases in particular aortic aneurysm, chemotherapy cardiotoxicity and atherosclerosis can be considered.

Other indications are also considered, such as transplant rejection, autoimmune diseases (other than Rheumatoid Arthritis, e.g. Inflammatory Bowel Disease . . . ) or neurodegenerative diseases.

However, the preparation of the ^{99m}Tc-rhAnnexin V-128 is rather complicated because of various problems.

The cysteine present in the rhAnnexin V-128 can easily lead to dimerization due to the formation of a disulfide bridge between two cysteines, thus decreasing the chemical purity of the preparation.

Moreover the protein is subjected to protein aggregation, a physical phenomenon in which misfolded proteins aggregate.

It should be noted that rhAnnexin V-128 undergoes several stressful process steps (freeze-thawing, bulk formulation, lyophilization), as well as long-term storage and reconstitution with radioactive ^{99m}TcO₄— solution, during which aggregation is likely to occur. For these reasons the chemical purity is an analytical parameter which has to be carefully monitored in this preparation.

The radiochemical purity is also a critical parameter for radiopharmaceuticals. It is essential both for safety reasons and also for its technical/diagnostic performance. The labeling with ^{99m}TcO₄— occurs through a series of reactions/equilibriums involving REDOX reactions and trans-chelation, and several radiochemical impurities may form if the conditions are not optimal.

Therefore, the recombinant protein rhAnnexin V-128 is sensitive, can flocculate during freeze-thawing, can dimerize through the free SH groups of the Cys, both during the thawing process, the manufacturing process and also during radiolabelling. A specific formulation is necessary for creating suitable conditions leading to a final ^{99m}Tc-rhAnnexin V-128 preparation with high radiochemical and chemical purity, and high stability.

In Tait et al. “Structural Requirements for In Vivo Detection of Cell Death with ^{99m}Tc-Annexin V”; The Journal of Nuclear Medicine—Vol. 46—No. 5—pp. 807-815 (2005) the labelling of rhAnnexin V-128 protein with ^{99m}Tc is described.

However, Tait's work describes a two vials kit approach (with rhAnnexin V-128 in the form of a solution), that requires a final purification to obtain the ^{99m}Tc-rhAnnexin V-128. This is not an optimal formulation, as the final purification is an important drawback in routinely clinical practices because it requires a rather complex manipulation of the product, with associated radio safety and sterility issues and not compatible with pharmaceutical quality standards.

The Patent Applications CN 103159842 and Lu C. et al. “Kit formulation for ^{99m}Tc-labeling of recombinant Annexin V molecule with a C-terminally engineered cysteine”; J Radioanal Nucl Chem—Vol. 304—pp. 571-578 (2015) describe a preparation method for the labeling of a different form of Annexin V which is modified on the C-terminus.

The described method comprises mainly two steps: 1) preparation of a phosphate buffer solution (pH 7.4) containing the protein, glucoheptonate and EDTA as transitional ligands, Stannous salt and diluted hydrochloric acid; 2) ^{99m}TcO₄ Na is added to the solution in step 1 and the so-obtained solution is mixed in water batch at 35-37° C. to give the labeled product ^{99m}Tc-Cys-Annexin V.

However, the Annexin V according to CN 103159842 differs from the present Annexin V-128 since it presents a single Cysteine residue on the C-terminal, which is able to bind Tc-99m, while it lacks the modified N-terminus, with the addition of six amino acids (among which there is one Cys); moreover, in Annexin V-128 the Cys that was present in position 316 has been substituted by a Serine, thus subtracting a residue that favors dimerization via disulphide bonds.

The process described in the above said patent and article is performed at pH=7.4 and therefore it could not be applied to a process involving Annexin V-128 since this pH favors dimer formation.

Moreover, in the Patent Applications CN 103159842 and in the publication by Lu et al., above reported, there is no mention of any characterization of the chemical purity and of any antioxidant agent for preventing dimerization, which on the contrary is critical for Annexin V-128 according to the present invention.

From the above said it is evident the necessity to develop a new composition comprising Annexin V-128 allowing an easier labeling process and leading to a final ^{99m}Tc-labeled rhAnnexin V-128 formulation with high chemical and radiochemical purity, and high stability.

This labeling process should be based on direct reconstitution of a pre-formulated single vial possibly without requiring any additional filtration or purification step prior to injection.

### BRIEF DESCRIPTION OF THE FIGURES

FIG. 1 reports a schematic view of the process for the preparation of the lyophilized rhAnnexin V-128 according to the invention

FIG. 2 shows SPECT images of ^{99m}Tc-rhAnnexin V-128 uptake in front paws of a healthy mouse (a) and in a mouse model of collagen induced arthritis (b); (c) shows the uptake in the CIA mouse after treatment with an anti-inflammatory drug (no ^{99m}Tc-rhAnnexin V-128 uptake detectable anymore).

### SUMMARY OF THE INVENTION

It is described a composition comprising lyophilized rhAnnexin V-128 suitable for the preparation of ^{99m}Technetium formulation for intravenous administration.

### DETAILED DESCRIPTION OF THE INVENTION

The present invention allows to overcome the above said problems thanks to a lyophilized composition suitable for intravenous administration comprising Annexin V-128 in combination with suitable excipients, including particularly an antioxidant agent, in a pH range of 5.0-6.6.

Moreover the present application refers also to a formulation obtained by adding to the above said composition a suitable volume of eluate from a commercial ^{99m}TcO₄— generator.

Preferably the lyophilized composition as above defined includes also a specific buffer.

The antioxidant is included with the purpose of decreasing the Annexin V-128 dimer content both during the long-term storage of the lyophilized composition and also in the ^{99m}Tc-rhAnnexin V-128 preparation after labeling. In the absence of the antioxidant the dimer formation was not under control, leading to a non-adequate chemical purity.

The addition of the antioxidant agent allowed to limit the dimer formation and ensure a dimer content below 10% during the long-term storage of the lyophilized composition and also for at least 6 h after radiolabeling.

Therefore, according to the present invention, the antioxidant has a key role in maintain a good level of chemical purity and it is not added with the purpose of preventing the re-oxidation of reduced technetium to pertechnetate. Different antioxidants, such as sodium metabisulfite, nicotinamide, pyridoxine hydrochloride, a-tocopherol acetate, monothioglycerol, were evaluated.

Besides the use of the antioxidant, the control of dimerization and therefore of the chemical purity, is also accomplished by keeping the pH within the range 5.0-6.6. Higher pH values favor the dimer formation, while at lower pH values opalescence of the solution was observed, probably due to a decrease in protein solubility.

Regarding the choice of the buffer, initially the use of citrate buffer was attempted, as this is the buffer in which Annexin V-128 is currently stored. However, a radiochemical purity around 85-90% could only be achieved. With the purpose of shifting the radiochemical purity value above 90%, which is the commonly accepted lower limit for radiopharmaceutical preparations, different buffers, such as lactate, succinate, glycolic, TRIS and histidine, were evaluated. The lactate buffer was chosen, as it allowed to reach radiochemical purity values consistently around 94-96%.

The use of the antioxidant and of the specific buffer allowed also to obtain a single-vial lyophilized composition with a long-term stability of at least 18 months. As a reference, the lyophilized composition described in the previous art (Lu et al., 2015) is declared to be stable for 210 days.

According to the present invention, the antioxidant and the specific buffer mentioned above are included in lyophilized composition comprising also:
a reducing agent;
a transchelating agent;
a lyoprotectant and cake-forming agent

Optionally, the composition can also include a radiation stability enhancer and/or a solubilizer.

In the lyophilized composition according to the invention the above said components are normally present in the following quantities:
antioxidant agent (with the purpose of achieving a high chemical purity): above 0.005 mg/vial
buffer: pH comprised between 5.0 and 6.6, with a concentration above 10 mM—reducing agent: above 0.005 mg/vial;
transchelating agent: above 0.02 mg/vial;
lyoprotectant and cake-forming agent: above 10 mg/vial

Optionally, the composition can also include a radiation stability enhancer in a quantity above 0.005 mg/vial and/or a solubilizer in a quantity above 1 mg/vial Several tests were performed during the development in order to define the above-described composition. Initially, the rhAnnexin V-128 lyophilized composition was prepared in presence of citrate buffer, including the following components:
rhAnnexin V-128 (active pharmaceutical ingredient)
stannous chloride (reducing agent)
sodium a-D-Glucoheptonate dihydrate (transchelating agent)
gentisic acid sodium salt hydrate (radiation stability enhancer)
hydroxyproplyl-p-cyclodextrin (solubilizer)
trehalose dihydrate (lyoprotectant and cake-forming agent)
pH=5.4

The quantities of each component were slightly modified with the purpose of optimizing the formulation. The various batches of this lyophilized composition, after radiolabeling, gave a radiochemical purity that was never above 90% (as determined by both ITLC and HPLC) and a stability not longer than 1.5 hours.

In a second step, the rhAnnexin V-128 lyophilized composition was prepared in presence of lactate buffer, instead of citrate, at different pH values (up to 6.4), in the absence of antioxidant agent. The so-obtained composition, after radiolabeling, gave an improved radiochemical purity (well above 90% by both ITLC and HPLC). However, in these tests it was also observed that pH increase favors dimer formation, the dimer percentage increasing more rapidly at higher pH, after radiolabeling. In a third step, the presence antioxidant agent and lactate buffer were tested. A lyophilized composition similar to the previous one was prepared, at a pH of 5.8, including also an antioxidant (sodium metabisulfite). After radiolabeling, the radiochemical purity was confirmed to be well above 90% and the chemical purity was remarkably improved, giving values around 97-98% (by SEC-HPLC and RP-HPLC) for at least 6 h after radiolabeling.

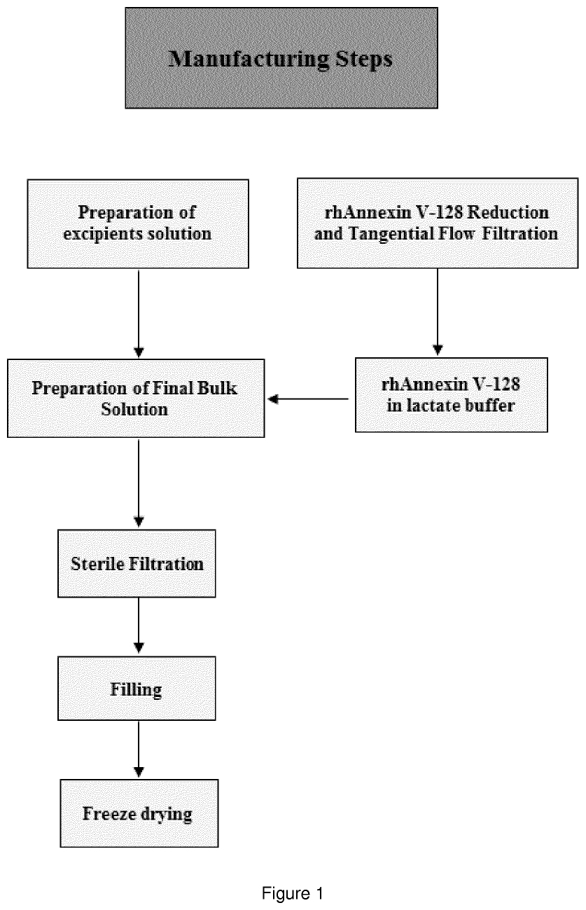

A lyophilized composition according to the present invention can be prepared according to a process comprising the following steps (see also FIG. 1):
Thawing of the frozen rhAnnexin V-128 at a controlled temperature (5° C.±3° C.)—Reduction by the addition of an antioxidant/reducing agent,
Buffer exchange by tangential flow filtration, to substitute the buffer in which the rhAnnexin V-128 is supplied (normally citrate buffer) with the buffer as above defined;
Preparation of the excipient bulk solution (consisting of: antioxidant agent, transchelating agent, radiation stability enhancer, solubilizer, lyoprotectant/cake-forming agent as above defined)
addition of the required amount of rhAnnexin V-128 solution to the excipient bulk solution
Dispensation of the bulk solution into vials and lyophilization.

The final ^{99m}Tc-rhAnnexin V-128 formulation suitable for intravenous administration can be prepared by adding to the above said composition a suitable volume of eluate from a commercial ^{99m}TcO₄— generator containing up to 740 MBq of radioactivity and keeping the vial under slight rotation for 90 min at room temperature.

It is worth noticing that the so-prepared ^{99m}Tc-rhAnnexin V-128 formulation maintains a high chemical and radiochemical purity, as determined by ITLC, SEC-HPLC and RP-HPLC for at least 6 hours.

The essential features of the composition/formulation according to the invention as well as of their process of preparation as above described are the use of an antioxidant agent and the use of a specific buffer, in the pH range 5.0-6.6.

These features allow to obtain an injectable formulation with a high radiochemical purity (^{99m}Tc-rhAnnexin V-128 monomer by SEC-HPLC and ITLC 90%) and high chemical purity (rhAnnexin V-128 monomer by SEC-HPLC and RP-HPLC 90%, dimers 10%), which are maintained for at least 6 h after radiolabelling.

The lyophilized composition according to the invention has a shelf life of at least 18 months at 2-8° C., and can be radiolabeled at room temperature, giving a high chemical and radiochemical purity and good stability for at least 6 hours after labeling, with a controlled dimer percentage.

The composition according to the invention makes the rhAnnexin V-128 available as a lyophilized single vial product that needs just to be reconstituted with a ^{99m}TcO₄— solution eluted from a commercially available generator without the need for any final purification.

Moreover, thanks to the fact that the radiolabeling procedure has been validated, only a limited quality control check (radiochemical purity analysis by ITLC) is required at the hospital level prior injection. The formulation according to the invention was tested for its diagnostic performance in several animal models (liver apoptosis, collagen induced arthritis model, endocarditis/myocarditis, inflammatory bowel disease, and others).

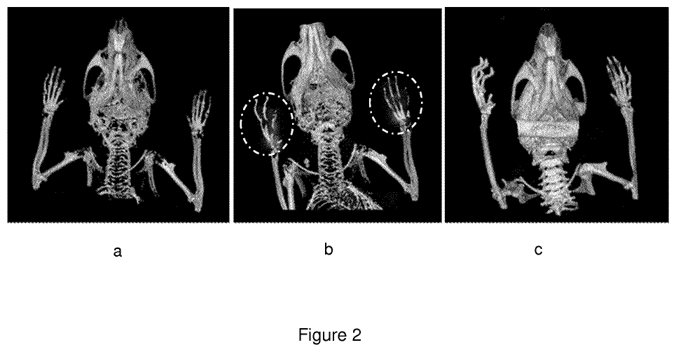

An example of SPECT images in a collagen induced arthritis model is shown as FIG. 2.

Annexin V-128 was also tested for its toxicity in a complete preclinical toxicology package (designed in accordance with the relevant guidelines and with the input received from regulatory agencies), in 15 days repeated dose toxicology studies in rodent and non-rodent species. A cytokine release assay was also included in the preclinical package. The outcome of these studies showed that Annexin V-128 has a very favorable safety profile (data can be provided if needed).

The formulation was tested for its safety and biodistribution in a Phase I study in human volunteers and is currently being tested in Phase II studies in Rheumatology and Cardiovascular indications.

### Example 1

Preparation of a Lyophilized rhAnnexin V-128 Composition Suitable for the Preparation of ^{99m}Tc-rhAnnexin V-128 Formulation for Intravenous Administration

### Composition:

rhAnnexin V-128 (0.4 mg);
stannous chloride (0.01 mg);
sodium a-D-Glucoheptonate dihydrate (3 mg)
gentisic acid sodium salt hydrate (0.02 mg)
hydroxypropyl-p-cyclodextrin (5 mg)
sodium metabisulfite (0.02 mg)
trehalose dihydrate (50 mg)
lactate buffer 150 mM, pH 5.8

### Preparation:

rhAnnexin V-128 was thawed and introduced into a tangential flow filtration system in order to exchange the buffer. Metabisulfite was also introduced at this step. This filtration procedure was carried on until at least 7 diavolumes of formulation buffer have been exchanged. The dimer content was checked by SEC-HPLC (maximum acceptable value=5%), and the protein concentration was also assessed (acceptable range 1-2 mg/mL). At the end of the filtration procedure, the solution was brought to a final concentration of 1 mg/mL of protein.

The other excipients were all dissolved in water for injection, in appropriate amounts:
D(+)-Trehalose dehydrate (powder): an appropriate amount was weighed to obtain a concentration of 50 mg/ml in the final bulk solution;
Sodium α-D-Glucoheptonate dihydrate (stock solution of 60 mg/ml): appropriate volume is added to obtain a concentration of 3 mg/ml in the final bulk solution;
Gentisic acid sodium salt hydrate (stock solution of 0.1 mg/ml): appropriate volume is added to obtain a concentration of 0.02 mg/ml in the final bulk solution;
Stannous Chloride dehydrate (stock solution of 1 mg/ml): appropriate volume is added to obtain a concentration of 0.01 mg/ml in the final bulk solution.

The final bulk solution was prepared adding an appropriate volume of rhAnnexin V-128 solution to an appropriate volume of excipient bulk solution.

The final bulk solution was filtered (0.22 μm filter) and automatically filled into the vials (1 mL/vial) and lyophilized.

### Example 2

Preparation of ^{99m}Tc-rhAnnexin V-128 Starting from Lyophilized Composition Described in Example 1
The vial cap was flipped off and the vial was placed in a suitable radiation shield;
2 mL of Sodium Pertechnetate Tc-99m solution containing 740 MBq of radioactivity were aseptically added to the vial in the lead shield;
The vial was removed from the lead shield and placed in an appropriately shielded roller. The vial was kept under slight rotation for 90 min at room temperature:
The vial was removed from the shielded roller and placed again in a lead shield; A sample of the so-obtained solution was withdrawn and analyzed for its chemical and radiochemical purity (SEC-HPLC, ITLC) immediately after radiolabelling and also after 6 h, and the following results were obtained. These data have been obtained from three different batches.

| | | | | STANDARD |
|---|---|---|---|---|
| | | ACCEPTANCE | AVERAGE | DEVIATION |
| TEST | METHOD | CRITERIA | (n = 3) | (n = 3) |
| Radiochemical Purity (% ^{99m}Tc- | ITLC | ≥90.0% | 98.6 | 0.6 |
| rhAnnexin V-128) | T = 0 | | | |
| Radiochemical Purity (% ^{99m}TcO₄ + | | ≤8.0% | 0.9 | 0.9 |
| ^{99m}TcO₂) | | | | |
| Radiochemical Purity (% ^{99m}Tc- | | ≤10.0% | 0.5 | 0.2 |
| glucoheptonate) | | | | |
| Radiochemical Purity (% ^{99m}Tc- | ITLC | ≥90.0% | 98.2 | 0.5 |
| rhAnnexin V-128) | T = 0 + 6 h | | | |
| Radiochemical Purity (% ^{99m}TcO₄ + | | ≤8.0% | 1.4 | 0.6 |
| ^{99m}TcO₂) | | | | |
| Radiochemical Purity (% ^{99m}Tc- | | ≤10.0% | 0.4 | 0.1 |
| glucoheptonate) | | | | |
| Radiochemical Purity (% ^{99m}Tc- | SEC-HPLC | ≥90.0% | 96.8 | 0.5 |
| rhAnnexin V-128) | T = 0 | | | |
| Radiochemical Purity (% ^{99m}Tc- | SEC-HPLC | ≥90.0% | 96.6 | 0.4 |
| rhAnnexin V-128) | T = 0 + 6 h | | | |

The above said formulation can be used as a diagnostic tool and also for selecting the best treatment as well as for monitoring medical treatment efficacy in rheumatology (for example rheumatoid arthritis, Axial Spondyloarthritis), cardiovascular diseases (as for example aortic aneurysm, chemotherapy cardiotoxicity, endocarditis and myocarditis) atherosclerosis (in particular for the detection and staging of atherosclerotic plaque), oncology, transplant rejection, autoimmune diseases, neurology, and other pathologies having as a hallmark the process of apoptosis and/or as marker of treatment response for treatment-induced apoptosis.

1. A lyophilized composition suitable for intravenous administration comprising rhAnnexin V-128 in combination with an antioxidant agent, in a pH range of 5.0-6.6.

2. The lyophilized composition according to claim 1 wherein the antioxidant agent is chosen among: sodium metabisulfite, nicotinamide, pyridoxine hydrochloride, a-tocopherol acetate, monothioglycerol.

3. The lyophilized composition according to claim 1, comprising also a buffer, chosen among lactate buffer, succinate buffer, glycolic buffer, TRIS, histidine buffer.

4. A lyophilized composition according to claim 1, suitable for intravenous administration, comprising:
an antioxidant agent,
a buffer having a pH comprised between 5.0-6.6;
a reducing agent;
a transchelating agent;
a lyoprotectant and cake-forming agent;
and possibly a radiation stability enhancer and/or a solubilizer.

5. The lyophilized composition according to claim 4 wherein said components are present in the following quantities:
the antioxidant agent in a quantity above 0.005 mg/vial
the buffer having a pH comprised between 5.0-6.6 with a concentration above 10 mM;
the reducing agent, in a quantity above 0.005 mg/vial;
the transchelating agent, in a quantity above 0.02 mg/vial;
the lyoprotectant and cake-forming agent, in a quantity above 10 mg/vial the radiation stability enhancer and the solubilizer, if present, in a quantity above 0.005 mg/vial and above 1 mg/vial respectively.

6. A process for preparing a lyophilized formulation according to claim 1 comprising the following steps:
thawing of the frozen rhAnnexin V-128;
adding an antioxidant/reducing agent,
buffer exchange by tangential flow filtration, to substitute the buffer in which the rhAnnexin V-128 is supplied with a buffer chosen among lactate buffer, succinate buffer, glycolic buffer, TRIS, and histidine buffer;
preparation of the excipient bulk solution including: transchelating agent, radiation stability enhancer, solubilizer, antioxidant agent, lyoprotectant and cake-forming agent;
addition of the required volume of rhAnnexin V-128 solution to the excipient bulk solution;
dispensation of the bulk solution into vials and lyophilization.

7. A formulation suitable for intravenous administration comprising a ^{99m}Tc-rhAnnexin V-128 obtained by reacting a single-vial lyophilized rhAnnexin V-128 formulation according to claim 1 with an eluate from a commercial ^{99m}Tc04-generator.

8. A process for obtaining a ^{99m}Tc-rhAnnexin V-128 according to claim 7 comprising the following steps:
adding a suitable volume of eluate from a commercial ^{99m}Tc04-generator containing up to 740 MBq of radioactivity to the vial containing the lyophilized formulation; and
rotating the vial for 90 minutes at room temperature.

9-13. (canceled)

14. A method for monitoring treatment efficacy of a disease in a subject, comprising the administration of the formulation according to claim 7 to the subject, wherein the disease is selected in the group consisting of rheumatic diseases, cardiovascular diseases, oncology, transplant rejection, autoimmune diseases, neurologic diseases and atherosclerosis.

15. A method of diagnosing a disease in a subject comprising the administration of the formulation according to claim 7, wherein said disease is selected in the group consisting of rheumatic diseases, cardiovascular diseases, oncology, transplant rejection, autoimmune diseases, neurologic diseases and atherosclerosis.

16. The method according to claim 15, wherein said cardiovascular diseases are selected from the group consisting of aortic aneurysm, chemotherapy cardiotoxicity, endocarditis and myocarditis.

17. The diagnostic method according to claim 15, wherein said rheumatic diseases are selected from the group consisting of rheumatoid arthritis and Axial Spondyloarthritis.

18. The diagnostic method according to claim 15, wherein said disease is atherosclerosis, and said diagnostic method detects and stages atherosclerotic plaque.

19. The method of claim 14, wherein the subject is a human subject.

20. The method of claim 15, wherein the subject is a human subject.
